(19) United States
(12) Patent Application Publication
(10) Pub. No.: US 2007/0123449 A1
(43) Pub. Date: May 31, 2007

(54) High load particles for inhalation having rapid release properties

(75) Inventors: Henry Brush, Arlington, MA (US); Fen-Ni Fu, Northborough, MA (US); Michael M. Lipp, Framingham, MA (US); Jennifer L. Schmitke, Boston, MA (US)

Correspondence Address: ELMORE PATENT LAW GROUP, PC, 209 MAIN STREET, N. CHELMSFORD, MA, 01863, US

(73) Assignee: Advanced Inhalation Research, Inc., Cambridge, MA (US)

(21) Appl. No.: 11/590,562

(22) Filed: Oct. 31, 2006

Related U.S. Application Data
(60) Provisional application No. 60/732,238, filed on Nov. 1, 2005.

(51) Int. Cl. A61K 38/28; A61K 31/19

(52) U.S. Cl. 514/3; 514/574

(57) ABSTRACT

The invention generally relates to formulations having particles comprising phospholipids, bioactive agent and excipients and the pulmonary delivery thereof. Dry powder inhaled insulin formulations are disclosed. Improved formulations comprising DPPC, insulin and sodium citrate which are useful in the treatment of diabetes are disclosed. Also, the invention relates to a method of for the pulmonary delivery of a bioactive agent comprising administering to the respiratory tract of a patient in need of treatment, or diagnosis an effective amount of particles comprising a bioactive agent or any combination thereof in association, wherein release of the agent from the administered particles occurs in a rapid fashion.

### RELATED APPLICATION

This application claims the benefit of U.S. Provisional Application No. 60/732,238, filed on Nov. 1, 2005. The entire teaching of the above application is incorporated herein by reference.

### BACKGROUND OF THE INVENTION

Pulmonary delivery of bioactive agents, for example, therapeutic, diagnostic and prophylactic agents, provides an attractive alternative to, for example, oral, transdermal and parenteral administration. That is, pulmonary administration can typically be completed without the need for medical intervention (self-administration), the pain often associated with injection therapy is avoided, and the amount of enzymatic and pH mediated degradation of the bioactive agent, frequently encountered with oral therapies, can be significantly reduced. In addition, the lungs provide a large mucosal surface for drug absorption and there is no first-pass liver effect of absorbed drugs. Further, it has been shown that high bioavailability of many molecules, for example, macromolecules, can be achieved via pulmonary delivery or inhalation. Typically, the deep lung, or alveoli, is the primary target of inhaled bioactive agents, particularly for agents requiring systemic delivery.

The release kinetics or release profile of a bioactive agent into the local and/or systemic circulation is a key consideration in most therapies, including those employing pulmonary delivery. That is, many illnesses or conditions require administration of a constant or sustained level of a bioactive agent to provide an effective therapy. Typically, this can be accomplished through a multiple dosing regimen or by employing a system that releases the medicament in a sustained fashion.

Delivery of bioactive agents to the pulmonary system, however, can result in rapid release of the agent following administration. For example, U.S. Pat. No. 5,997,848 to Patton et al. describes the absorption of insulin following administration of a dry powder formulation via pulmonary delivery. The peak insulin level was reached in about 30 minutes for primates and in about 20 minutes for human subjects. Further, Heinemann, Traut and Heise teach in Diabetic Medicine (14:63-72 (1997)) that the onset of action after inhalation reached half-maximal action in about 30 minutes, assessed by glucose infusion rate in healthy volunteer.

Diabetes mellitus is the most common of the serious metabolic diseases affecting humans. It may be defined as a state of chronic hyperglycemia, i.e., excess sugar in the blood, that results from a relative or absolute lack of insulin action. Insulin is a peptide hormone produced and secreted by B cells within the islets of Langerhans in the pancreas. Insulin promotes glucose utilization, protein synthesis, and the formation and storage of neutral lipids. It is generally required for the entry of glucose into muscle. Glucose, or “blood sugar,” is the principal source of carbohydrate energy for man and many other organisms. Excess glucose is stored in the body as glycogen, which is metabolized into glucose as needed to meet bodily requirements.

The hyperglycemia associated with diabetes mellitus is a consequence of both the underutilization of glucose and the overproduction of glucose from protein due to relatively depressed or nonexistent levels of insulin. Diabetic patients frequently require daily, usually multiple, injections of insulin that may cause discomfort. This discomfort leads many type 2 diabetic patients to refuse to use insulin injections, even when they are indicated.

A need exists for formulations suitable for efficient inhalation comprising bioactive agents, for example, insulin, and wherein the bioactive agent of the formulation is released in a manner that is at least as efficient as presently available treatments and prophylactics, especially for the treatment of diabetes. Such formulations allow patients the freedom of self titration leading to better self management of blood glucose levels.

A need also exists for formulations suitable for delivery to the lung and rapid release into the systemic and/or local circulation. Such formulations are expected to increase the willingness of patients to comply with prescribed therapy, and may achieve improved disease treatment and control.

A need also exists for formulations suitable for efficient inhalation, wherein the bioactive agent, for example insulin, is delivered into the lung at a high load and wherein the bioactive agent is more robust and stable than commercially available counterparts.

### SUMMARY OF THE INVENTION

Formulations having particles comprising, by weight, at least about 30% (for example between approximately 10% to approximately 30%) DPPC; between approximately 60% to approximately 90% (preferably between 60% and 70%) insulin; and approximately 10 and approximately 30% (such as approximately 10% to approximately 20%) sodium citrate are disclosed. In a preferred embodiment, the particles comprise, by weight, approximately 25% DPPC, approximately 60% insulin and approximately 15% sodium citrate.

The present invention also features methods for treating a human patient in need of insulin comprising administering pulmonary to the respiratory tract of a patient in need of treatment, an effective amount of particles comprising by weight, approximately 25% DPPC, approximately 60% insulin and approximately 15% sodium citrate, wherein release of the insulin is rapid. This method is particularly useful for the treatment of diabetes. If desired, the particles can be delivered in a single, breath actuated step.

The invention also features a kit comprising two or more receptacles comprising unit dosages selected from the insulin formulations described herein. For example, the formulation can be particles comprising, by weight, approximately 20% DPPC, approximately 60% insulin and approximately 20% sodium citrate; or comprising, by weight, approximately 25% DPPC, approximately 60% insulin and approximately 15% sodium citrate; or comprising, by weight, approximately 30% DPPC, approximately 60% insulin and approximately 10% sodium citrate or comprising by weight, approximately 10% DPPC, approximately 70% insulin and approximately 20% sodium citrate; or comprising, by weight, approximately 15% DPPC, approximately 70% insulin and approximately 15% sodium citrate; or comprising, by weight, approximately 20% DPPC, approximately 70% insulin and approximately 10% sodium citrate Combinations of receptacles containing different formulations within the same kit are also a feature of the present invention. For example, the kit can comprise two or more receptacles comprising unit dosages of particles comprising 10% to 30% DPPC, 60% to 70% insulin and 10% to 20% sodium citrate and one or more receptacles comprising unit dosages of particles comprising, by weight, 10% to 30% DPPC, 60% to 70 % insulin and 10% to 20% sodium citrate. In another embodiment, the kit comprises one or more receptacles comprising unit dosages of particles comprising 25% DPPC, 60% insulin and 15% sodium citrate and one or more receptacles comprising unit dosages of particles comprising, by weight, 10% DPPC, 70% insulin and 20% sodium citrate.

The present invention also features a kit comprising at least two receptacles each receptacle containing a different amount of dry powder insulin suitable for inhalation.

In another embodiment, the above-described particles comprise a mass of from about 0.5 mg to about 20 mg of insulin (for example, 0.5, 1.0, 1.5, 2.5, 5, 7.5, 10, 12.5, 15, 17.5, 20, or 25 mg). In a preferred embodiment, the above-described particles comprise a mass of 4.3 mg of insulin. In yet another preferred embodiment, the above-described particles comprise a mass of 3.9 mg of insulin. In another embodiment, the above-described particles have a tap density less than about 0.4 g/cm³, preferably less than 0.1 g/cm³ and/or a median geometric diameter of from between about 2 micrometers and about 30 micrometers and/or an aerodynamic diameter of from about 1 micrometer to about 5 micrometers.

The invention has numerous advantages. For example, particles suitable for inhalation can be designed to possess a controllable, in particular a rapid, release profile. This rapid release profile provides for abbreviated residence of the administered bioactive agent, in particular insulin, in the lung and decreases the amount of time in which therapeutic levels of the agent are present in the local environment or systemic circulation. The rapid release of agent provides a desirable alternative to injection therapy currently used for many therapeutic, diagnostic and prophylactic agents requiring rapid release of the agent, such as insulin for the treatment of diabetes. The formulation of the present invention has the unexpected discovery that a formulation comprising fewer excipients allows for a more robust and stable bioactive agent, for example insulin. In addition, the invention provides a method of delivery to the pulmonary system wherein the high initial release of agent typically seen in inhalation therapy is boosted, giving very high initial release. Consequently, patient compliance and comfort can be increased by not only reducing frequency of dosing, but by providing a therapy that is more amenable to patients.

This dry powder delivery system allows for efficient dose delivery from a small, convenient and inexpensive delivery device. In addition, the simple and convenient inhaler together with the room temperature stable powder may offer an attractive replacement for currently available injections. This system has the potential to help achieve improved glycaemic control in patients with diabetes by increasing the willingness of patients to comply with insulin therapy.

### DETAILED DESCRIPTION OF THE INVENTION

The invention relates to particles capable of releasing bioactive agent, in particular insulin, in a rapid fashion. Methods of treating disease and delivery via the pulmonary system using these particles is also disclosed. As such, the particles possess rapid release properties. “Rapid release,” as that term is used herein, refers to an increased pharmacodynamic response (including, but not limited to serum levels of the bioactive agent and glucose infusion rates) typically seen in the first two hours following administration, and more preferably in the first hour. Rapid release also refers to a release of active agent, in particular inhaled insulin, in which the period of release of an effective level of agent is at least the same as, preferably shorter than that seen with presently available subcutaneous injections of active agent, in particular, insulin lispro and regular soluble insulin.

In one embodiment, the rapid release particles are formulated using insulin, sodium citrate and a phospholipid. It is believed that the selection of the appropriate phospholipid affects the release profile as described in more detail below. In a preferred embodiment, the rapid release is characterized by both the period of release being shorter and the levels of agent released being greater.

Drug release rates can be described in terms of the half-time of release of a bioactive agent from a formulation. As used herein the term “half-time” refers to the time required to release 50% of the initial drug payload contained in the particles. Fast or rapid drug release rates generally are less than 30 minutes and range from about 1 minute to about 60 minutes.

In one embodiment, the particles include one or more phospholipids in place of the DPPC described above. The phospholipid or combination of phospholipids is selected to impart specific drug release properties to the particles. Phospholipids suitable for pulmonary delivery to a human subject are preferred. In one embodiment, the phospholipid is endogenous to the lung. In another embodiment, the phospholipid is non-endogenous to the lung.

The phospholipid can be present in the particles in an amount ranging from about 0 to about 35 weight %. Preferably, it can be present in the particles in an amount ranging from about 10 to about 30 weight %, for example, 10%, 15%, 20%, 25% or 30%.

Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids, for example, phospholipids having their head group modified, e.g., alkylated or polyethylene glycol (PEG)-modified, also can be employed. Preferably, the phospholipid is DPPC.

In a preferred embodiment, the phospholipid is selected to maintain an equivalent matrix transition temperature of the particles with DPPC, which is related to the phase transition temperature, as defined by the melting temperature (Tₘ), the crystallization temperature (T_{c}) and the glass transition temperature (T_{g}) of the phospholipid or combination of phospholipids employed in forming the particles. Tₘ, T_{c} and T_{g} are terms known in the art. For example, these terms are discussed in Phospholipid Handbook (Gregor Cevc, editor, 1993, Marcel-Dekker, Inc.).

Phase transition temperatures for phospholipids or combinations thereof can be obtained from the literature. Sources listing phase transition temperatures of phospholipids include, for instance, the Avanti Polar Lipids (Alabaster, Ala.) Catalog or the Phospholipid Handbook (Gregor Cevc, editor, 1993, Marcel-Dekker, Inc.). Small variations in transition temperature values listed from one source to another may be the result of experimental conditions such as moisture content.

Experimentally, phase transition temperatures can be determined by methods known in the art, in particular by differential scanning calorimetry. Other techniques to characterize the phase behavior of phospholipids or combinations thereof include synchrotron X-ray diffraction and freeze fracture electron microscopy.

The amounts of phospholipids to be used to form particles having a desired or targeted matrix transition temperature can be determined experimentally, for example, by forming mixtures in various proportions of the phospholipids of interest, measuring the transition temperature for each mixture, and selecting the mixture having the targeted transition temperature. The effects of phospholipid miscibility on the matrix transition temperature of the phospholipid mixture can be determined by combining a first phospholipid with other phospholipids having varying miscibilities with the first phospholipid and measuring the transition temperature of the combinations.

Combinations of one or more phospholipids with other materials also can be employed to achieve a desired matrix transition temperature. Examples include polymers and other biomaterials, such as, for instance, lipids, sphingolipids, cholesterol, surfactants, polyaminoacids, polysaccharides, proteins, salts and others. Amounts and miscibility parameters selected to obtain a desired or targeted matrix transition temperatures can be determined as described above.

The particles of the instant invention, in particular the rapid release particles, are delivered pulmonarily. “Pulmonary delivery,” as that term is used herein refers to delivery to the respiratory tract. The “respiratory tract,” as defined herein, encompasses the upper airways, including the oropharynx and larynx, followed by the lower airways, which include the trachea followed by bifurcations into the bronchi and bronchioli (e.g., terminal and respiratory). The upper and lower airways are called the conducting airways. The terminal bronchioli then divide into respiratory bronchioli which then lead to the ultimate respiratory zone, namely, the alveoli, or deep lung. The deep lung, or alveoli, are typically the desired target of inhaled therapeutic formulations for systemic drug delivery.

“Pulmonary pH range,” as that term is used herein, refers to the pH range which can be encountered in the lung of a patient. Typically, in humans, this range of pH is from about 6.4 to about 7.0, such as from 6.4 to about 6.7 pH values of the airway lining fluid (ALF) have been reported in “Comparative Biology of the Normal Lung”, CRC Press, (1991) by R. A. Parent and range from 6.44 to 6.74.

Therapeutic, prophylactic or diagnostic agents, can also be referred to herein as “bioactive agents,” “medicaments” or “drugs.” The amount of therapeutic, prophylactic or diagnostic agent present in the particles can range from about 60 weight percent to about 90 weight percent, for example, 60%, 65%, 70%, 75%, 85% or 90%. In this invention, the preferred agent is insulin, e.g., human insulin and includes Humulin® Lente® (Humulin® L; human insulin zinc suspension), Humulin® R (regular soluble insulin (RI)), Humulin® Ultralente® (Humulin-U), and Humalog® 100 (insulin lispro (IL)) from Eli Lilly Co. (Indianapolis, Ind.; 100 U/mL).

The particles can further comprise a carboxylic acid which is distinct from the agent and lipid, in particular a phospholipid. In one embodiment, the carboxylic acid includes at least two carboxyl groups. Carboxylic acids, include the salts thereof as well as combinations of two or more carboxylic acids and/or salts thereof. In a preferred embodiment, the carboxylic acid is a hydrophilic carboxylic acid or salt thereof. Suitable carboxylic acids include but are not limited to hydroxydicarboxylic acids, hydroxytricarboxylic acids and the like. Citric acid and citrates, such as, for example, sodium citrate, are preferred. Combinations or mixtures of carboxylic acids and/or their salts also can be employed.

The carboxylic acid can be present in the particles in an amount ranging from about 0 weight % to about 80 weight %. Preferably, the carboxylic acid can be present in the particles in an amount of about 10% to about 20%, for example 5%, 10%, 15%, 20%, or 25%.

The particles, also referred to herein as powder, can be in the form of a dry powder suitable for inhalation. In a particular embodiment, the particles can have a tap density of less than about 0.4 g/cm³. Particles which have a tap density of less than about 0.4 g/cm³ (e.g., 0.4 g/cm³) are referred to herein as “aerodynamically light particles”. More preferred are particles having a tap density less than about 0.1 g/cm³ (e.g., 0.1 g/cm³).

Aerodynamically light particles have a preferred size, e.g., a volume median geometric diameter (VMGD) of at least about 5 microns (μm). In one embodiment, the VMGD is from about 2 μm to about 30 μm (for example, 2, 3, 4, 5, 10, 15, 20, 25 or 30 μm). In another embodiment of the invention, the particles have a VMGD ranging from about 9 μm to about 30 μm. In other embodiments, the particles have a median diameter, mass median diameter (MMD), a mass median envelope diameter (MMED) or a mass median geometric diameter (MMGD) of at least 5 μm, for example, from about 2 μm to about 30 μm (for example, 2, 3, 4, 5, 10, 15, 20, 25 or 30 μm), or from about 7 μm to about 8 μm (for example, 6 μm, 7 ∞m, or 8 μm).

Aerodynamically light particles preferably have “mass median aerodynamic diameter” (MMAD), also referred to herein as Aaerodynamic diameter”, between about 1 μm and about 5 μm (for example 1, 2, 3, 4, or 5 μm). In one embodiment of the invention, the MMAD is between about 1 μm and about 3 μm. In another embodiment, the MMAD is between about 3 μm and about 5 μm.

In another embodiment of the invention, the particles have an envelope mass density, also referred to herein as “mass density” of less than about 0.4 g/cm³. The envelope mass density of an isotropic particle is defined as the mass of the particle divided by the minimum sphere envelope volume within which it can be enclosed.

Tap density can be measured by using instruments known to those skilled in the art such as the Dual Platform Microprocessor Controlled Tap Density Tester (Vankel, N.C.) or a GeoPyc® instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which can contribute to low tap density include irregular surface texture and porous structure.

The diameter of the particles, for example, their VMGD, can be measured using an electrical zone sensing instrument such as a Multisizer Ile, (Coulter Electronic, Luton, Beds, England), or a laser diffraction instrument (for example, Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

In one embodiment, particles of the instant invention have an aerodynamic diameter of about 1.3 microns and a mean geometric diameter at 2 bar/16 mbar pressure of about 7.5 microns. In another embodiment, particles have about 44-45% of the particles with a fine particle fraction (FPF) less than about 3.4 microns, as detected using a 2 stage Anderson Cascade Impactor (ACI) assay. In another embodiment, particles have about 63-66% of the particles with a fine particle fraction of less than about 5.6 microns. Methods of measuring fine particle fraction using a 2 stage ACI assay are well known to those skilled in the art. One example of such an assay is as follows. Fine Particle Fractions (FPF) are measured using a reduced Thermo Anderson Cascade Impactor with two stages. Ten milligrams of powder are weighed into a size 2 hydroxpropyl methyl cellulose (HPMC) capsule. The powders are dispersed using a single-step, breath-actuated dry powder inhaler operated at 60 L/min for 2 seconds. The stages are selected to collect particles of an effective cutoff diameter (ECD) of (1) between 5.6 microns and 3.4 microns and (2) less than 3.4 microns and are fitted with porous filter material to collect the powder deposited. The mass deposited on each stage is determined gravimetrically. FPF is then expressed as a fraction of the total mass loaded into the capsule.

In another embodiment, particles of the instant invention have a mean geometric diameter at 1 bar of about 7 to about 8 microns as determined by RODOS. In another embodiment, particles have about 35% to about 40%, about 40% to about 45%, or about 45% to about 50% of the particles with a fine particle fraction of less than about 3.3 microns, as measured using a 3 stage ACI assay, as described herein.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

Suitable particles can be fabricated or separated, for example, by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be for example, between about 5 and about 30 μm, or optimally between about 5 and about 15 μm. In one preferred embodiment, at least a portion of the particles have a diameter between about 9 and about 11 μm. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 μm.

The particles can be prepared by spray drying. For example, a spray drying mixture, also referred to herein as “feed solution” or “feed mixture”, which includes the bioactive agent and one or more charged lipids having a charge opposite to that of the active agent upon association are fed to a spray dryer.

For example, when employing a protein active agent, the agent may be dissolved in a buffer system above or below the pI of the agent. Specifically, insulin, for example, may be dissolved in an aqueous buffer system (e.g., citrate, phosphate, acetate, etc.) or in 0.01 N HCl. The pH of the resultant solution then can be adjusted to a desired value using an appropriate base solution (e.g., 1 N NaOH). In one preferred embodiment, the pH may be adjusted to about pH 7.4. At this pH, insulin molecules have a net negative charge (pI=5.5). In another embodiment, the pH may be adjusted to about pH 4.0. At this pH, insulin molecules have a net positive charge (pI=5.5). In addition, if desired, the solutions can be heated to temperatures below their boiling points, for example, approximately 50 EC. Typically the cationic phospholipid is dissolved in an organic solvent or combination of solvents. The two solutions are then mixed together and the resulting mixture is spray dried.

Suitable organic solvents that can be present in the mixture being spray dried include, but are not limited to, alcohols, for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 50:50 to about 90:10. The mixture can have a neutral, acidic or alkaline pH. Optionally, a pH buffer can be included. Preferably, the pH can range from about 3 to about 10.

The total amount of solvent or solvents being employed in the mixture being spray dried generally is greater than about 98 weight percent. The amount of solids (drug, charged lipid and other ingredients) present in the mixture being spray dried can vary from about 1.0 weight percent to about 5 weight percent.

Using a mixture which includes an organic and an aqueous solvent in the spray drying process allows for the combination of hydrophilic and hydrophobic components, while not requiring the formation of liposomes or other structures or complexes to facilitate solubilization of the combination of such components within the particles.

Suitable spray-drying techniques are described, for example, by K. Masters in ASpray Drying Handbook,”John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of a suitable spray dryer using rotary atomization includes the Mobile Minor spray dryer, manufactured by Niro, Denmark. The hot gas can be, for example, air, nitrogen or argon.

Preferably, the particles of the invention are obtained by spray drying using an inlet temperature between about 100° C. and about 400° C. and an outlet temperature between about 50° C. and about 130° C.

The spray dried particles can be fabricated with a rough surface texture to reduce particle agglomeration and improve flowability of the powder. The spray-dried particle can be fabricated with features which enhance aerosolization via dry powder inhaler devices, and lead to lower deposition in the mouth, throat and inhaler device.

The particles of the invention can be employed in compositions suitable for drug delivery via the pulmonary system. For example, such compositions can include the particles and a pharmaceutically acceptable carrier for administration to a patient, preferably for administration via inhalation. The particles can be co-delivered with other similarly manufactured particles that may or may not contain yet another drug. Methods for co-delivery of particles is disclosed in U.S. patent application Ser. No. 09/878,146, filed Jun. 8, 2001, the entire teachings of which are incorporated herein by reference. The particles can also be co-delivered with larger carrier particles, not including a therapeutic agent, the latter possessing mass median diameters, for example, in the range between about 50 μm and about 100 μm. The particles can be administered alone or in any appropriate pharmaceutically acceptable carrier, such as a liquid, for example, saline, or a powder, for administration to the respiratory system.

Particles including a medicament, for example, one or more of drugs, are administered to the respiratory tract of a patient in need of treatment, prophylaxis or diagnosis. Administration of particles to the respiratory system can be by means such as those known in the art. For example, particles are delivered from an inhalation device. In a preferred embodiment, particles are administered via a dry powder inhaler (DPI). Metered-dose-inhalers (MDI), nebulizers or instillation techniques also can be employed.

Various suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No. 4,995,385 issued Feb. 26, 1991 to Valentini, et al., and U.S. Pat. No. 5,997,848 issued Dec. 7, 1999 to Patton, et al. Other examples include, but are not limited to, the Spinhaler (Fisons, Loughborough, U.K.), Rotahaler (Glaxo-Wellcome, Research Triangle Technology Park, N.C.), FlowCaps (Hovione, Loures, Portugal), Inhalator (Boehringer-Ingelheim, Germany), and the Aerolizer (Novartis, Switzerland), the diskhaler (Glaxo-Wellcome, RTP, NC) and others, such as those known to those skilled in the art. Preferably, the particles are administered as a dry powder via a dry powder inhaler.

In one embodiment, the dry powder inhaler is a simple, breath actuated device. An example of a suitable inhaler which can be employed is described in U.S. Pat. No.: 6,766,799 issued on Jul. 27, 2004. The entire contents of this application are incorporated by reference herein. This pulmonary delivery system is particularly suitable because it enables efficient dry powder delivery of small molecules, proteins and peptide drug particles deep into the lung. Particularly suitable for delivery are the unique porous particles, such as the insulin particles described herein, which are formulated with a low mass density, relatively large geometric diameter and optimum aerodynamic characteristics (Edwards et al., 1998). These particles can be dispersed and inhaled efficiently with a simple inhaler device, as low forces of cohesion allow the particles to deaggregate easily. In particular, the unique properties of these particles confer the capability of being simultaneously dispersed and inhaled.

In one embodiment, the volume of the receptacle is at least about 0.37 cm³. In another embodiment, the volume of the receptacle is at least about 0.48 cm³. In yet another embodiment, are receptacles having a volume of at least about 0.67 cm³ or 0.95 cm³. The invention is also drawn to receptacles which are capsules, for example, capsules designated with a particular capsule size, such as 2, 1, 0, 00 or 000. Suitable capsules can be obtained, for example, from Shionogi (Rockville, Md.). Blisters can be obtained, for example, from Hueck Foils, (Wall, N.J.). Other receptacles and other volumes thereof suitable for use in the instant invention are known to those skilled in the art.

The receptacle encloses or stores particles and/or respirable compositions comprising particles. In one embodiment, the particles and/or respirable compositions comprising particles are in the form of a powder. The receptacle is filled with particles and/or compositions comprising particles, as known in the art. For example, vacuum filling or tamping technologies may be used. Generally, filling the receptacle with powder can be carried out by methods known in the art. In one embodiment of the invention, the particles which are enclosed or stored in a receptacle have a mass of at least about 5 milligrams. In another embodiment, the mass of the particles stored or enclosed in the receptacle comprises a mass of bioactive agent from at least about 1.5 mg to at least about 20 milligrams.

Preferably, particles administered to the respiratory tract travel through the upper airways (oropharynx and larynx), the lower airways, which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung.

In one embodiment of the invention, delivery to the pulmonary system of particles is in a single, breath-actuated step, as described in U.S. Pat. No.: 6,858,199, issued on Feb. 22, 2005 and continuation-in-part of U.S. patent application Ser. No. 09/878,146, entitled, “Highly Efficient Delivery of a Large Therapeutic Mass Aerosol,” filed Jun. 8, 2001, the entire teachings of which are incorporated herein by reference. In one embodiment, the dispersing and inhalation occurs simultaneously in a single inhalation in a breath-actuated device. An example of a suitable inhaler which can be employed as described in U.S. Patent Publication 20040011360,. The entire contents of this application are incorporated by reference herein. In another embodiment of the invention, at least 50% of the mass of the particles stored in the inhaler receptacle is delivered to a subject's respiratory system in a single, breath-activated step.

In one further embodiment, at least 1.5 milligrams, or at least 5 milligrams, or at least 10 milligrams of a bioactive agent is delivered by administering, in a single breath, to a subjects respiratory tract particles enclosed in the receptacle. In a preferred embodiment 4.3 milligrams of a bioactive agent is delivered by administering, in a single breath, to a subjects respiratory tract particles enclosed in the receptacle. In yet another preferred embodiment, 3.9 milligrams of a bioactive agent is delivered by administering, in a single breath, to a subjects respiratory tract particles enclosed in the receptacle. Amounts of bioactive agent as high as 15 milligrams can be delivered.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations (e.g., by means of an appropriate, conventional pharmacological protocol). In one embodiment, depending upon the patient, the dosage range is from about 2 IU to about 40 IU of bioactive agent, in particular, insulin, per meal. As used herein 2 IU is equivalent to 0.9 milligram; 6 IU is equivalent to 2.6 milligrams; and 10 IU is equivalent to 3.9 milligrams in one preferred embodiment or to 4.3 milligrams in another preferred embodiment.

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

Drug release rates can be described in terms of release constants. The first order release constant can be expressed using the following equations:
M₍ₜ₎=M_{(∞)}*(131 e^{-k*t}) (1)
Where k is the first order release constant. M_{(∞)} is the total mass of drug in the drug delivery system, e.g. the dry powder, and M₍ₜ₎ is the amount of drug mass released from dry powders at time t.

Equations (1) may be expressed either in amount (i.e., mass) of drug released or concentration of drug released in a specified volume of release medium. For example, Equation (1) may be expressed as:
C₍ₜ₎=C_{(∞)}*(1−e^{-k*t}) or Release₍ₜ₎=Release_{(∞)}*(1−e^{k*t}) (2)
Where k is the first order release constant. C_{(∞)} is the maximum theoretical concentration of drug in the release medium, and C₍ₜ₎ is the concentration of drug being released from dry powders to the release medium at time t.

Drug release rates in terms of first order release constant can be calculated using the following equations:
k=−ln(M_{(∞)}−M₍ₜ₎/M_{(∞)}/t (3)

As used herein, the term “a” or “an” refers to one or more.

The term “nominal dose” as used herein, refers to the total mass of bioactive agent which is present in the mass of particles targeted for administration and represents the maximum amount of bioactive agent available for administration.

Applicants' technology is based upon pulmonary delivery of dry powder aerosols composed of large, porous particles wherein each individual particle is capable of comprising both drug and excipient within a porous matrix. The particles are geometrically large but have low mass density and aerodynamic size. This results in a powder that is easily dispersible. The ease of dispersibility of the dry powder aerosols of large porous particles described herein allows for efficient systemic delivery of protein therapeutics from simple, breath activated, capsule based inhalers.

The invention also features a kit comprising at least two receptacles, each receptacle containing a different amount of dry powder insulin suitable for inhalation. The powder can be, but is not limited to any such dry powder insulin as described herein. In addition, the invention also features a kit comprising two or more receptacles comprising two or more unit dosages comprising particles comprising the bioactive agent formulations described herein. Depending on the bioavailability of the bioactive agent in the formulation, the formulation can contain more bioactive agent than the amount that is delivered to the subjects bloodstream. For example, as described in the Examples section below, a unit dosage of 2 IU, 6 IU, 10 IU etc, can be contained in the receptacle administered to the subject, yet if the bioavailability is less than 100%, then only a portion of the bioactive agent reaches the subjects bloodstream.

In one embodiment, the bioactive agent is insulin. For example, the formulation can be particles comprising, by weight, approximately 20% DPPC, approximately 60% insulin and approximately 20% sodium citrate; or comprising, by weight, approximately 25% DPPC, approximately 60% insulin and approximately 15% sodium citrate; or comprising by weight, approximately 30% DPPC, approximately 60% insulin and approximately 10% sodium citrate; or comprising by weight, approximately 10% DPPC, approximately 70% insulin and approximately 20% sodium citrate; or comprising, by weight, approximately 15% DPPC, approximately 70% insulin and approximately 15% sodium citrate; or comprising by weight, approximately 20% DPPC, approximately 70% insulin and approximately 10% sodium citrate. The desired dose can be achieved in a number of different ways. For example, the size of the receptacle can be varied and/or the volume of formulation loaded into the receptacle and/or the formulation (e.g., percent of insulin) can be varied in order to achieve the desired dose. The desired dose can be the dose in the receptacle, or the dose that is bioavailable to the subject (e.g., the amount released into the subject's bloodstream). When the receptacle is only partially filled with the formulation, the remainder of the receptacle can remain empty or be loaded to 100% capacity with a filler.

The kits described herein can be used to deliver bioactive agents, for example, insulin to a subject in need of the bioactive agent. When the bioactive agent is insulin, the dose administered to the subject can be altered, for example, by a patient or by a medical provider, by increasing or decreasing the number of receptacles (e.g., capsules) of insulin containing particles, thereby increasing or decreasing the unit dosage of the insulin. When a patient is in need of a higher dose of insulin than usual, that patient can administer to himself or herself additional receptacles, or a different combination of receptacles, so that the dose of insulin is increased to the desired amount. Conversely, when a patient needs less insulin, the patient can administer to himself or herself fewer receptacles, or a different combination of receptacles, such that the dose is decreased to the desired amount. The kits may also contain instructions for the use of the reagents in the kits (e.g., the receptacles containing the formulation). Through the use of such kits, accurate dosing can be accomplished.

Exemplification

### EXAMPLE I

### Development Batch Data for Capsules, Human Insulin Inhalation Powder, 4.3 mg Insulin (High Dose Strength, Approximately Equivalent to 10 U Subcutaneous)

Two commercial system development lots of Capsules, Human Insulin Inhalation Powder, 4.3 mg Insulin were spray dried manufactured at the commercial manufacturing site (Alkermes Brickyard Square, Chelsea, Mass.), filled into capsules using commercial unit filler (G-100) with continuous dosator technology and packaged into ACLAR/Aluminum peel open blisters in an Aluminum overpouch. Emitted Dose, Aerodynamic Particle Size Distribution of the Emitted Dose, and Microscopic Evaluation of the Emitted Powder data were generated using the commercial design Insulin Inhaler (Part No. 9001017) manufactured using brass tools. Results indicated that the spray drying process conserved the integrity of the drug substance. Batch release data tables follow (Table 1).

TABLE 1
Batch Release Data of Human Insulin Inhalation Powder, 4.3 mg Insulin

| | | | Lot No. |
|---|---|---|---|
| | | Lot No. | EAS15AUG05 |
| | | EAS28JUL05 | 4.3 mg Insulin/capsule |
| | | 4.3 mg Insulin/capsule | MFG Date: August 2005 |
| | | MFG Date: July 2005 | MFG Site: BYS- |
| Test | | MFG Site: BYS-Chelsea | Chelsea |
| (Method | | Batch Size: | Batch Size: |
| Code) | Proposed Specification | 50,000 Capsules | 90,000 Capsules |
| Physical | Clear capsule containing | Practically white, no visible | Practically white, no |
| Appearance | white to practically white | foreign particulate | visible foreign particulate |
| 110-00722 | powder, no visible foreign | | |
| | particulate matter. | | |
| | Capsule has a black band and | | |
| | black printing on capsule that | | |
| | indicates dosage strength is | | |
| | 10 U (4.3 mg). | | |
| Identification | Retention time of the sample | Pass | Pass |
| 110-00710 | compares favorably to the | | |
| 110-00720 | retention time of the reference | | |
| | standard. | | |
| Insulin Assay | Label Strength (LS)¹ is 4.3 mg | 96.3% of LS | 101.5% of LS |
| 110-00930 | insulin. Mean of composite | (4.1 mg | (4.4 mg |
| | assay results is within 90.0-110.0% | Insulin/capsule) | Insulin/capsule) |
| | of the LS (3.9 to 4.7 mg | | |
| | insulin). | | |
| Content | Meets USP<905> | 90.8%, 94.1%, 89.2%, | 93.1%, 105.4%, 105.7%, |
| uniformity of | LS = 4.3 mg insulin | 93.1%, 96.0%, 91.4%, | 97.8%, 103.7%, 105.9%, |
| pre-metered | | 91.6%, 91.7%, 98.8%, | 103.1%, 89.4%, 101.0%, |
| dose | | 94.6% (% LS) | 100.3% (% LS) |
| 110-00930 | | Range: | Range: |
| | | 89.2-98.8% LS | 89.4-105.9% LS |
| | | RSD: 3.0 (%) | RSD: 5.6 (%) |
| Emitted Dose | Label claim (LC)² is 3.2 mg | Mean: 97% of LC | Mean: 104% of LC |
| 110-00920, | insulin. Mean of individual | (3.1 mg Insulin/capsule) | (3.3 mg Insulin/capsule) |
| 110-00930 | determinations is within 85 to | | |
| | 115% of the LC (2.7 to 3.7 mg | | |
| | insulin). | | |
| Content | Meets limits outlined in | 103%, 104%, 85%, 90%, | 92%, 94%, 103%, 109%, |
| uniformity of | USP<601>: “Dose | 90%, 102%, 95%, 89%, | 104%, 114%, 100%, |
| Emitted Dose | Uniformity over the Entire | 111%, 99% | 102%, 111%, 109% |
| 110-00920, | Contents” | Range: 85-111% | Range: 92-114% |
| 110-00930 | LC = 3.2 mg insulin | | |
| Aerodynamic | IP-S1: To be monitored | IP-S1 = 0.50 mg | IP-S1 = 0.55 mg |
| Particle Size | S2-S3: To be monitored | S2-S3 = 1.16 mg | S2-S3 = 1.25 mg |
| Distribution of | S4-S5 mean between 0.66 and | S4-S5 = 0.93 mg | S4-S5 = 0.97 mg |
| Emitted Dose: | 1.43 mg insulin | S6-SF = 0.04 mg | S6-SF = 0.03 mg |
| (mg | S6-SF: To be monitored | | |
| Insulin/capsule) | | | |
| 110-02254, | | | |
| 110-00928 | | | |
| High | NMT 1.5% | 0.1% | 0.1% |
| Molecular | | | |
| Weight Protein | | | |
| (HMWP) | | | |
| 110-00710, | | | |
| 110-00721 | | | |
| A-21 | NMT 2.0% | 0.6% | 0.7% |
| Desamido | | | |
| Insulin | | | |
| 110-00710, | | | |
| 110-00720 | | | |
| Other Related | NMT 3.0% | 0.1% | 0.1% |
| Substances | | | |
| 110-00710, | | | |
| 110-00720 | | | |
| Water Content | Not more than 10.0%. | 6.1% | 6.1% |
| 110-00711 | Alert if more than 7.5%. | | |
| Microscopic | Predominantly spheroid | Conforms | Conforms |
| Evaluation of | particles. | | |
| the Emitted | | | |
| Powder | | | |
| 110-00922, | | | |
| 110-01014 | | | |
| Microbial | Total Aerobic Count: | NT | Total Aerobic Count: |
| Limits | NMT 100 CFU per gram | | <7 CFU per gram |
| CSOP-AIR-1 | Combined Yeast and Mold: | | Combined Yeast and |
| Modified USP | NMT 10 CFU per gram | | Mold: |
| <61> | Staphylococcus aureus: | | <3 CFU per gram |
| | Absent | | Staphylococcus aureus: |
| | Pseudomonas aeruginosa: | | Absent |
| | Absent | | Pseudomonas |
| | | | aeruginosa: Absent |
| Residual | NMT 0.5% ethanol | ≦0.3% ethanol | 0.33% ethanol |
| Solvents | | | |
| 110-00728 | | | |

### EXAMPLE II

### Stability Data

Two development lots of 4.3 mg insulin strength HIIP capsules that are representative of the phase 1 configuration (EAS28JUL05 and EAS15AUG05) were evaluated at in-use stability conditions. These lots were manufactured and packaged at the Alkermes Chelsea, Mass. commercial manufacturing site. These representative stability lots are expected to be predictive of clinical lot stability for all future studies.

The HIIP capsules are packaged in both primary (blister) and secondary (aluminum pouch) critical packaging. The in-pouch, in-use storage program, where the HIIP capsules are stored in both primary and secondary critical packaging at 30° C./65% RH, evaluates the patient in-use period prior to the opening of secondary packaging. The out-of-pouch storage program, where the HIIP capsules are stored in primary packaging only at 30° C./65% RH, evaluates the patient in-use period subsequent to the opening of (and removal of the blister card from) secondary packaging.

Data presented in this section support that the 4.3 mg HIIP capsule strength will meet stability requirements at the patient in-use condition of 30° C./65% RH (30° C./65% RH_IP) for up to 6 weeks. Given that the stability performance of the 4.3 mg HIIP capsules over the patient in-use period is comparable or better than that of the other two HIIP dosage strengths (0.9 and 2.6 mg), the data herein is considered supportive of a 12-month refrigerated storage period prior to use.

TABLE 2
Overview of Available Human Insulin Inhalation Powder Supporting
Commercial System Stability Data

| | | | |
|---|---|---|---|
| | | Completed | Date of |
| Batch Number | Storage Conditions | Test Intervals | Manufacture¹ |

4.3 mg Insulin Capsule Strength

| | | | |
|---|---|---|---|
| EAS28JUL05 | 30° C./65% RH_IP | 2, 4, 6 weeks | July 2005 |
| | 30° C./65% RH_OOP | 1, 2, 3 weeks | |
| EAS15AUG05 | 30° C./65% RH_IP | 2, 4, 6 weeks | August 2005 |
| | 30° C./65% RH_OOP | 1, 2, 3 weeks | |

### EXAMPLE III

### Tabulated Stability Data

Stability data for the drug product are provided in the following tables (Tables 3-6). The data tables show the scheduled time points, which may vary from the actual analysis dates. Any statistical calculations are performed using the actual sample age. The actual dates and ages are recorded in a stability database.

TABLE 3
In-use, In Pouch Stability (30° C./65% RH)
In-Pouch Storage Stability at 30° C./65% RH for Supporting Lot EAS28JUL05
of Human Insulin Inhalation Powder Capsules, 4.3 mg Insulin Strength

| Analytical Property | 0 | 2 weeks | 4 weeks | 6 weeks |
|---|---|---|---|---|
| Emitted Dose (% LC) | Mean: 97% of LC | NT | NT | Mean: 94% of LC |
| | (3.1 mg | | | (3.0 mg |
| | Insulin/capsule) | | | Insulin/capsule) |
| Content Uniformity | 103%, 104%, 85%, | NT | NT | 103%, 102%, 89%, |
| of the Emitted Dose¹ | 90%, 90%, 102%, | | | 95%, 84%, 92%, |
| (% LC) | 95%, 89%, 111%, | | | 96%, 90%, 101%, |
| | 99% | | | 91% |
| | Range: 85-111% | | | Range: 84-103% |
| Aerodynamic Particle | IP-S1 = 0.50 mg | NT | NT | IP-S1 = 0.61 mg |
| Size Distribution of | S2-S3 = 1.16 mg | | | S2-S3 = 1.26 mg |
| Emitted Dose | S4-S5 = 0.93 mg | | | S4-S5 = 1.03 mg |
| (mg insulin) | S6-SF = 0.04 mg | | | S6-SF = 0.03 mg |
| Insulin Assay (% LS) | 96.3% of LS | 95.9% | 92.4% | 94.7% |
| | (4.1 mg | (4.1 mg | (4.0 mg | (4.1 mg |
| | Insulin/capsule) | Insulin/capsule) | Insulin/capsule) | Insulin/capsule) |
| High Molecular | 0.1% | 0.2% | 0.3% | 0.3% |
| Weight Protein | | | | |
| (HMWP) (%) | | | | |
| A21-Desamido | 0.6% | 0.5% | 0.6% | 0.7% |
| Insulin (%) | | | | |
| Other Related | 0.1% | 0.2% | 0.5% | 0.7% |
| Substances (%) | | | | |
| Water Content (%) | 6.1% | 6.0% | 5.9% | 6.1% |
| Microscopic | Conforms | NT | NT | Conforms |
| Evaluation of the | | | | |
| Emitted Powder | | | | |

LS = Label Strength
LC = Label Claim
NT = Not Tested (Indicated test not scheduled for this time point)

TABLE 4
In-Pouch Storage Stability at 30° C./65% RH
In-Pouch Storage Stability at 30° C./65% RH for Supporting Lot EAS15AUG05
of Human Insulin Inhalation Powder Capsules, 4.3 mg Insulin Strength

| Analytical Property | 0 | 2 weeks | 4 weeks | 6 weeks |
|---|---|---|---|---|
| Emitted Dose (% LC) | Mean: 104% of LC | NT | NT | Mean: |
| | (3.3 mg | | | 101% of |
| | Insulin/capsule) | | | LC |
| | | | | (3.2 mg |
| | | | | Insulin/capsule) |
| Content Uniformity of the | 92%, 94%, 103%, | NT | NT | 94%, |
| Emitted Dose¹ (% LC) | 109%, 104%, 114%, | | | 100%, |
| | 100%, 102%, 111%, | | | 98%, |
| | 109% | | | 100%, |
| | Range: 92-114% | | | 111%, |
| | | | | 108%, |
| | | | | 105%, |
| | | | | 87%, |
| | | | | 108%, |
| | | | | 104103% |
| | | | | Range: 87-111% |
| Aerodynamic Particle Size | IP-S1 = 0.55 mg | NT | NT | IP-S1 = 0.58 mg |
| Distribution of Emitted | S2-S3 = 1.25 mg | | | S2-S3 = 1.41 mg |
| Dose | S4-S5 = 0.97 mg | | | S4-S5 = 1.02 mg |
| (mg insulin) | S6-SF = 0.03 mg | | | S6-SF = 0.03 mg |
| Insulin Assay (% LS) | 101.5% of LS | 97.6% | 100.4% | 93.9% |
| | (4.4 mg | (4.2 mg | (4.3 mg | (4.0 mg |
| | Insulin/capsule) | Insulin/capsule) | Insulin/capsule) | Insulin/capsule) |
| High Molecular Weight | 0.1% | 0.2% | 0.2% | 0.3% |
| Protein (HMWP) (%) | | | | |
| A21-Desamido Insulin | 0.7% | 0.4% | 0.5% | 0.6% |
| (%) | | | | |
| Other Related Substances | 0.1% | 0.2% | 0.5% | 0.6% |
| (%) | | | | |
| Water Content (%) | 6.1% | 6.0% | 5.7% | 6.0% |
| Microscopic Evaluation of | Conforms | NT | NT | Conforms |
| the Emitted Powder | | | | |

LS = Label Strength
LC = Label Claim
NT = Not Tested (Indicated test not scheduled for this time po

[symbol]

TABLE 5
In-use, Out of Pouch Stability (30° C./65% RH)
Out-of-Pouch Storage Stability at 30° C./65% RH for Supporting Lot EAS28JUL05 of
Human Insulin Inhalation Powder Capsules, 4.3 mg Insulin Strength

| Analytical Property | 0 | 1 week | 2 week | 3 week |
|---|---|---|---|---|
| Emitted Dose (% LC) | Mean: 97% of | NT | Mean: 95% of LC | NT |
| | LC | | (3.0 mg Insulin/capsule) | |
| | (3.1 mg | | | |
| | Insulin/capsule) | | | |
| Content Uniformity of the | 103%, 104%, | NT | 96%, 103%, 87%, 88%, | NT |
| Emitted Dose | 85%, 90%, 90%, | | 90%, 89%, 97%, 115%, | |
| (% LC) | 102%, 95%, | | 91%, 93% | |
| | 89%, 111%, 99% | | Range: 87-115% | |
| | Range: 85-111% | | | |
| Aerodynamic Particle Size | IP-S1 = 0.50 mg | NT | IP-S1 = 0.55 mg | NT |
| Distribution of Emitted | S2-S3 = 1.16 mg | | S2-S3 = 1.31 mg | |
| Dose (mg insulin) | S4-S5 = 0.93 mg | | S4-S5 = 1.03 mg | |
| | S6-SF = 0.04 mg | | S6-SF = 0.03 mg | |
| Insulin Assay (% LS) | 96.3% of LS | 94.9% | 99.6% | 95.6% |
| | (4.1 mg | (4.1 mg | (4.3 mg | (4.1 mg |
| | Insulin/capsule) | Insulin/capsule) | Insulin/capsule) | Insulin/capsule) |
| High Molecular Weight | 0.1% | 0.2% | 0.2% | 0.3% |
| Protein (HMWP, %) | | | | |
| A-21 Desamido Insulin (%) | 0.6% | 0.6% | 0.5% | 0.5% |
| Other Related Substances | 0.1% | 0.2% | 0.2% | 0.2% |
| (%) | | | | |
| Water Content (%) | 6.1% | 6.4% | 6.6% | 6.8% |
| Microscopic Evaluation of | Conforms | NT | Conforms | NT |
| the Emitted Powder | | | | |

LS = Label Strength
LC = Label Claim
NT = Not Tested (Indicated test not scheduled for this time point)

TABLE 6
Out-of-Pouch Storage Stability at 30° C./65% RH
Out-of-Pouch Storage Stability at 30° C./65% RH for Supporting Lot EAS15AUG05 of
Human Insulin Inhalation Powder Capsules, 4.3 mg Insulin Strength

| Analytical Property | 0 | 1 week | 2 week | 3 week |
|---|---|---|---|---|
| Emitted Dose (% LC) | Mean: 104% of LC | NT | Mean: 98% of LC | NT |
| | (3.3 mg | | (3.1 mg | |
| | Insulin/capsule) | | Insulin/capsule) | |
| Content Uniformity of the | 92%, 94%, 103%, | NT | 95% 94, 106%, | NT |
| Emitted Dose | 109%, 104%, 114%, | | 110%, 102%, | |
| (% LC) | 100%, 102%, 111%, | | 98%, 98% 97, | |
| | 109% | | 72%, 97%, 98%, | |
| | Range: 92-114% | | 106105% | |
| | | | Range: 72-110% | |
| Aerodynamic Particle Size | IP-S1 = 0.55 mg | NT | IP-S1 = 0.63 mg | NT |
| Distribution of Emitted Dose | S2-S3 = 1.25 mg | | S2-S3 = 1.42 mg | |
| (mg insulin) | S4-S5 = 0.97 mg | | S4-S5 = 1.06 mg | |
| | S6-SF = 0.03 mg | | S6-SF = 0.04 mg | |
| Insulin Assay (% LS) | 101.5% of LS | 98.1% | 101.2% | 97.5% |
| | (4.4 mg | (4.2 mg | (4.4 mg | (4.2 mg |
| | Insulin/capsule) | Insulin/capsule) | Insulin/capsule) | Insulin/capsule) |
| High Molecular Weight | 0.1% | 0.2% | 0.2% | 0.2% |
| Protein (HMWP, %) | | | | |
| A-21 Desamido Insulin (%) | 0.7% | 0.4% | 0.5% | 0.4% |
| Other Related Substances (%) | 0.1% | 0.1% | 0.2% | 0.2% |
| Water Content (%) | 6.1% | 6.2% | 6.3% | 6.6% |
| Microscopic Evaluation of the | Conforms | NT | Conforms | NT |
| Emitted Powder | | | | |

LS = Label Strength
LC = Label Claim
NT = Not Tested (Indicated test not scheduled for this time point)

### EXAMPLE IV

### Development Batch Data for Capsules, Human Insulin Inhalation Powder, 3.9 mg Insulin (High Dose Strength, Approximately Equivalent to 10 U Subcutaneous)

Three commercial system development lots (Lot #s Fill19, Fill22 and Fill31) of Capsules, Human Insulin Inhalation Powder, 3.9 mg Insulin were manufactured via spray-drying at the commercial manufacturing site (Alkermes Brickyard Square, Chelsea, Mass.), filled into capsules using a commercial unit filler (G-100) with continuous dosator technology and packaged into ACLAR/Aluminum peel open blisters in an Aluminum overpouch. Emitted Dose, Aerodynamic Particle Size Distribution of the Emitted Dose, and Microscopic Evaluation of the Emitted Powder data were generated using the commercial design Insulin Inhaler (Part No. 9001017) manufactured using brass tools. Results indicated that the spray drying process conserved the integrity of the drug substance. Capsule lot batch release data is shown in Tables 1 and 2.

TABLE 7
3.9 mg Insulin/Capsule Release Data

RELEASE DATA

| ATTRIBUTE | | | | Process |
|---|---|---|---|---|
| Product Lot | Fill19 | Fill22 | Fill31 | Mean |
| FPF<3.3 | 1.25 | 1.14 | 1.09 | 1.16 |
| FPF<4.7 | 2.11 | 2.04 | 1.93 | 2.03 |
| Total Mass | 2.93 | 2.85 | 2.67 | 2.82 |
| (mg insulin) | | | | |
| Total Mass | 94 | 92 | 86 | 91 |
| (% LC) | | | | |
| Emitted Dose | 3.1 | 3.1 | 3.2 | 3.1 |
| (mg insulin) | | | | |
| Emitted Dose | 99 | 99 | 102 | 100 |
| (% LC) | | | | |
| Water Content | 6.5 | 6.4 | 6.4 | 6.4 |
| A-21 (%) | 0.5 | 0.5 | 0.6 | 0.5 |
| ORS (%) | 0.1 | 0.1 | 0.1 | 0.1 |
| HMWP (%) | 0.1 | 0.1 | 0.1 | 0.1 |
| Assay (mg | 4.0 | 4.0 | 3.8 | 3.9 |
| insulin) | | | | |
| Assay (% LS) | 103.4 | 103.6 | 98.6 | 101.9 |

TABLE 8
3.9 mg Insulin/Capsule 10 U aPSD Stage Grouping Results
10U APSD STAGE GROUPING RESULTS

| POWDER/CAPSULE | | | | |
|---|---|---|---|---|
| LOT NO. | | Fill19^{†} | Fill22^{†} | Fill31 |
| MEAN | IP-S1 | 0.46 (1.3) | 0.43 (0.42) | 0.38 (15) |
| (mg insulin) | S2 | 0.35 (4.5) | 0.38 (4.2) | 0.36 (17) |
| | S3 | 0.86 (4.0) | 0.90 (3.9) | 0.85 (12) |
| | S4-S5 | 1.14 (4.0) | 1.05 (1.2) | 1.02 (12) |
| | S6-SF | 0.11 (11) | 0.09 (3.7) | 0.07 (32) |
| | FPM | 2.11 (3.8) | 2.04 (1.9) | 1.93 (12) |
| | <4.7 | | | |
| | FPM | 1.25 (4.6) | 1.14 (1.4) | 1.09 (13) |
| | <3.3 | | | |
| | % LC | 94 (8.1) | 92 (8.6) | 86 (10) |
| RANGE (mg | IP-S1 | 0.46-0.47 | 0.42-0.43 | 0.27-0.45 |
| insulin) | S2 | 0.34-0.37 | 0.37-0.40 | 0.28-0.45 |
| | S3 | 0.82-0.89 | 0.87-0.94 | 0.72-1.00 |
| | S4-S5 | 1.09-1.18 | 1.04-1.06 | 0.83-1.19 |
| | S6-SF | 0.10-0.12 | 0.09-0.09 | 0.04-0.10 |
| | FPM | 2.02-2.17 | 2.00-2.07 | 1.63-2.25 |
| | <4.7 | | | |
| | FPM | 1.19-1.31 | 1.12-1.15 | 0.86-1.28 |
| | <3.3 | | | |
| | % LC | 81-110 | 78-108 | 75-98 |

^{†}Reported results are a mean of three method executions
( ) Represents values in % RSD

What is claimed is:

1. A formulation having particles comprising by weight, 0% to 30% DPPC, 60% to 90% insulin and 10% to 20% sodium citrate.

2 A formulation having particles comprising, by weight, 20% DPPC, 60% insulin and 20% sodium citrate.

3. A formulation having particles comprising, by weight, 25% DPPC, 60% insulin and 15% sodium citrate.

4. A formulation having particles comprising, by weight, 30% DPPC, 60% insulin and 10% sodium citrate.

5. A formulation having particles comprising, by weight, 10% DPPC, 70% insulin and 20% sodium citrate.

6. A formulation having particles comprising, by weight, 15% DPPC, 70% insulin and 15% sodium citrate.

7. A formulation having particles comprising, by weight, 20% DPPC, 70% insulin and 10% sodium citrate.

8. The formulation of claim 1, wherein the particles comprise a mass of from about 1.5 mg to about 20 mg of insulin.

9. The formulation of claim 1, wherein the particles comprise a mass of about 4.3 mg of insulin per receptacle.

10. The formulation of claim 1, wherein the particles comprise a mass of about 3.9 mg of insulin per receptacle.

11. The formulation of claim 1, wherein the particles have a tap density less than about 0.4 g/cm³.

12. The formulation of claim 1, wherein the particles have a tap density less than about 0.1 g/cm³.

13. The formulation of claim 1, wherein the particles have a median geometric diameter of from about 2 micrometers to about 30 micrometers.

14. A method for treating a human patient in need of insulin comprising administering pulmonarily to the respiratory tract of a patient in need of treatment, an effective amount of particles comprising by weight, 25% DPPC, 60% insulin and 15% sodium citrate, wherein release of the insulin is rapid.

15. The method of claim 14, wherein the patient in need of treatment has diabetes mellitus.

16. The method of claim 14, wherein the particles have a mass of from about 1.5 mg to about 20 mg of insulin.

17. The method of claim 14, wherein the particles comprise a mass of about 4.3 mg of insulin per receptacle.

18. The method of claim 14, wherein the particles comprise a mass of about 3.9 mg of insulin per receptacle.

19. The method of claim 14, wherein the particles have a tap density less than about 0.4 g/cm³.

20. The method of claim 14, wherein the particles have a tap density less than about 0.1 g/cm³.

21. The method of claim 14, wherein the particles have a median geometric diameter of from about 2 micrometers to about 30 micrometers.

22. The method of claim 14, wherein administering the particles pulmonary includes delivery of the particles to the deep lung.

23. The method of claim 14, wherein administering the particles pulmonary includes delivery of the particles to the upper airways.

24. A method of delivering an effective amount of insulin to the pulmonary system, comprising:
a) providing a mass of particles comprising by weight, 25% DPPC, 60% insulin and 15 % sodium citrate; and
b) administering via simultaneous dispersion and inhalation the particles, from a receptacle having the mass of the particles, to a human subjects respiratory tract, wherein release of the insulin is rapid.

25. The method of claim 24, wherein the particles comprise a mass of from about 1.5 mg to about 20 mg of insulin.

26. The method of claim 24, wherein the particles comprise a mass of about 4.3 mg of insulin per receptacle.

27. The method of claim 24, wherein the particles have a tap density less than about 0.4 g/cm³.

28. The method of claim 24, wherein the particles have a tap density less than about 0.1 g/cm³.

29. The method of claim 24, wherein the particles have a median geometric diameter of from about 2 micrometers to about 30 micrometers.

30. The method of claim 24, wherein delivery to the pulmonary system includes delivery to the deep lung.

31. A kit for administration of insulin comprising two or more receptacles, wherein said receptacles comprise unit dosages selected from the group consisting of:
a) particles comprising, by weight, 20% DPP C, 60% insulin and 20% sodium citrate;
b) particles comprising, by weight, 25% DPPC, 60% insulin and 15% sodium citrate;
c) particles comprising, by weight, 30% DPPC, 60% insulin and 10% sodium citrate;
d) particles comprising, by weight, 10% DPP C, 70% insulin and 20% sodium citrate;
e) particles comprising, by weight, 15% DPP C, 70% insulin and 15% sodium citrate; and
f) particles comprising, by weight, 20% DPP C, 70% insulin and 10% sodium citrate.

32. The kit of claim 31, wherein said kit further comprises instructions for use of said two or more receptacles.
